(19) United States
(12) Patent Application Publication
(10) Pub. No.: US 2023/0030343 A1
(43) Pub. Date: Feb. 2, 2023

(54) METHODS AND SYSTEMS FOR USING MULTI VIEW POSE ESTIMATION

(71) Applicant: BODY VISION MEDICAL LTD., Ramat Hasharon (IL)

(72) Inventors: Dima SEZGANOV, Ramat Hasharon (IL); Tomer AMIT, Ramat Hasharon (IL)

(21) Appl. No.: 17/814,576

(22) Filed: Jul. 25, 2022

Related U.S. Application Data
(63) Continuation of application No. PCT/IB2021/000027, filed on Jan. 25, 2021.
(60) Provisional application No. 62/965,628, filed on Jan. 24, 2020.

(51) Int. Cl. G16H 30/40; G16H 40/60

(52) U.S. Cl. CPC G16H 30/40; G16H 40/60; A61B 34/20

(57) ABSTRACT

A method includes receiving a sequence of medical images captured by a medical imaging device while the medical imaging device travels through a rotation, and showing an area of interest including a plurality of landmarks; determining a pose of each of a subset of the sequence of medical images in which the landmarks are visible; estimating a trajectory of the medical imaging device based on the determined poses of the subset and a trajectory constraint of the imaging device; determining a pose of one of the medical images in which the landmarks are not visible by extrapolating based on an assumption of continuity of movement of the medical imaging device; and determining a volumetric reconstruction for the area of interest based at least on at least some of the poses of the subset and the pose of the one of the medical images in which the landmarks are not visible.

### CROSS REFERENCE TO RELATED APPLICATION

This is a continuation application of International Application No. PCT/IB2021/000027, filed Jan. 25, 2021, which relates to and claims priority to commonly-owned, co-pending U.S. Provisional Patent Application Serial No. 62/965,628, filed on Jan. 24, 2020 and entitled “METHODS AND SYSTEMS FOR USING MULTI VIEW POSE ESTIMATION,” the contents of which are incorporated herein by reference in their entirety.

### FIELD OF THE INVENTION

The embodiments of the present invention relate to interventional devices and methods of use thereof.

### BACKGROUND OF INVENTION

Use of minimally invasive procedures such as endoscopic procedures, video-assisted thoracic surgery, or similar medical procedures can be used as diagnostic tool for suspicious lesions or as treatment means for cancerous tumors.

### SUMMARY OF INVENTION

In some embodiments, the present invention provides a method, comprising:
obtaining a first image from a first imaging modality,
extracting at least one element from the first image from the first imaging modality,
   wherein the at least one element comprises an airway, a blood vessel, a body cavity, or any combination thereof;
obtaining, from a second imaging modality, at least (i) a first image of a radiopaque instrument in a first pose and (ii) a second image of the radiopaque instrument in a second pose,
   wherein the radiopaque instrument is in a body cavity of a patient; generating at least two augmented bronchograms,
   wherein a first augmented bronchogram corresponds to the first image of the radiopaque instrument in the first pose, and
   wherein a second augmented bronchogram corresponds to the second image of the radiopaque instrument in the second pose,
determining mutual geometric constraints between:
   (i) the first pose of the radiopaque instrument, and
   (ii) the second pose of the radiopaque instrument,
estimating the first pose of the radiopaque instrument and the second pose of the radiopaque instrument by comparing the first pose of the radiopaque instrument and the second pose of the radiopaque instrument to the first image of the first imaging modality,
   wherein the comparing is performed using:
      (i) the first augmented bronchogram,
      (ii) the second augmented bronchogram, and
      (iii) the at least one element, and
   wherein the estimated first pose of the radiopaque instrument and the estimated second pose of the radiopaque instrument meets the determined mutual geometric constraints,
generating a third image; wherein the third image is an augmented image derived from the second imaging modality which highlights an area of interest,
wherein the area of interest is determined from data from the first imaging modality.

In some embodiments, the at least one element from the first image from the first imaging modality further comprises a rib, a vertebra, a diaphragm, or any combination thereof. In some embodiments, the mutual geometric constraints are generated by:
a. estimating a difference between (i) the first pose and (ii) the second pose by comparing the first image of the radiopaque instrument and the second image of the radiopaque instrument,
   wherein the estimating is performed using a device comprising a protractor, an accelerometer, a gyroscope, or any combination thereof, and wherein the device is attached to the second imaging modality;
b. extracting a plurality of image features to estimate a relative pose change,
   wherein the plurality of image features comprise anatomical elements, non-anatomical elements, or any combination thereof,
   wherein the image features comprise: patches attached to a patient, radiopaque markers positioned in a field of view of the second imaging modality, or any combination thereof,
   wherein the image features are visible on the first image of the radiopaque instrument and the second image of the radiopaque instrument;
c. estimating a difference between (i) the first pose and (ii) the second pose by using a at least one camera,
   wherein the camera comprises: a video camera, an infrared camera, a depth camera, or any combination thereof,
   wherein the camera is at a fixed location,
   wherein the camera is configured to track at least one feature,
   wherein the at least one feature comprises: a marker attached the patient, a marker attached to the second imaging modality, or any combination thereof, and
   tracking the at least one feature;
d. or any combination thereof.

In some embodiments, the method further comprises: tracking the radiopaque instrument for: identifying a trajectory, and using the trajectory as a further geometric constraint, wherein the radiopaque instrument comprises an endoscope, an endo-bronchial tool, or a robotic arm.

In some embodiments, the present invention is a method, comprising:
generating a map of at least one body cavity of the patient,
   wherein the map is generated using a first image from a first imaging modality, obtaining, from a second imaging modality, an image of a radiopaque instrument comprising at least two attached markers,
   wherein the at least two attached markers are separated by a known distance, identifying a pose of the radiopaque instrument from the second imaging modality relative to a map of at least one body cavity of a patient,
identifying a first location of the first marker attached to the radiopaque instrument on the second image from the second imaging modality,
identifying a second location of the second marker attached to the radiopaque instrument on the second image from the second imaging modality, and
measuring a distance between the first location of the first marker and the second location of the second marker,
projecting the known distance between the first marker and the second marker,
comparing the measured distance with the projected known distance between the first marker and the second marker to identify a specific location of the radiopaque instrument inside the at least one body cavity of the patient.

In some embodiments, the radiopaque instrument comprises an endoscope, an endo-bronchial tool, or a robotic arm.

In some embodiments, the method further comprises: identifying a depth of the radiopaque instrument by use of a trajectory of the radiopaque instrument.

In some embodiments, the first image from the first imaging modality is a pre-operative image. In some embodiments, the at least one image of the radiopaque instrument from the second imaging modality is an intra-operative image.

In some embodiments, the present invention is a method, comprising:
obtaining a first image from a first imaging modality,
extracting at least one element from the first image from the first imaging modality,
   wherein the at least one element comprises an airway, a blood vessel, a body cavity or any combination thereof;
obtaining, from a second imaging modality, at least (i) a one image of a radiopaque instrument and (ii) another image of the radiopaque instrument in two different poses of second imaging modality
   wherein the first image of the radiopaque instrument is captured at a first pose of second imaging modality,
   wherein the second image of the radiopaque instrument is captured at a second pose of second imaging modality, and
   wherein the radiopaque instrument is in a body cavity of a patient;
generating at least two augmented bronchograms correspondent to each of two poses of the imaging device, wherein a first augmented bronchogram derived from the first image of the radiopaque instrument and the second augmented bronchogram derived from the second image of the radiopaque instrument,
determining mutual geometric constraints between:
   (i) the first pose of the second imaging modality, and
   (ii) the second pose of the second imaging modality,
estimating the two poses of the second imaging modality relatively to the first image of the first imaging modality, using the correspondent augmented bronchogram images and at least one element extracted from the first image of the first imaging modality;
   wherein the two estimated poses satisfy the mutual geometric constrains.
generating a third image; wherein the third image is an augmented image derived from the second imaging modality highlighting the area of interest, based on data sourced from the first imaging modality.

In some embodiments, anatomical elements such as: a rib, a vertebra, a diaphragm, or any combination thereof, are extracted from the first imaging modality and from the second imaging modality.

In some embodiments, the mutual geometric constraints are generated by:
a. estimating a difference between (i) the first pose and (ii) the second pose by comparing the first image of the radiopaque instrument and the second image of the radiopaque instrument,
   wherein the estimating is performed using a device comprising a protractor, an accelerometer, a gyroscope, or any combination thereof, and wherein the device is attached to the second imaging modality;
b. extracting a plurality of image features to estimate a relative pose change,
   wherein the plurality of image features comprise anatomical elements, non-anatomical elements, or any combination thereof,
   wherein the image features comprise: patches attached to a patient, radiopaque markers positioned in a field of view of the second imaging modality, or any combination thereof,
   wherein the image features are visible on the first image of the radiopaque instrument and the second image of the radiopaque instrument;
c. estimate a difference between (i) the first pose and (ii) the second pose by using a at least one camera,
   wherein the camera comprises: a video camera, an infrared camera, a depth camera, or any combination thereof,
   wherein the camera is at a fixed location,
   wherein the camera is configured to track at least one feature,
      wherein the at least one feature comprises: a marker attached the patient, a marker attached to the second imaging modality, or any combination thereof, and
   tracking the at least one feature;
d. or any combination thereof.

In some embodiments, the method further comprises tracking the radiopaque instrument to identify a trajectory and using such trajectory as additional geometric constrains, wherein the radiopaque instrument comprises an endoscope, an endo-bronchial tool, or a robotic arm.

In some embodiments, the present invention is a method to identify the true instrument location inside the patient, comprising:
using a map of at least one body cavity of a patient generated from a first image of a first imaging modality,
obtaining, from a second imaging modality, an image of the radiopaque instrument with at least two markers attached to it and having the defined distance between them,
that may be perceived from the image as located in at least two different body cavities inside the patient,
obtaining the pose of the second imaging modality relative to the map identifying a first location of the first marker attached to the radiopaque instrument on the second image from the second imaging modality,
identifying a second location of the second marker attached to the radiopaque instrument on the second image from the second imaging modality, and
measuring a distance between the first location of the first marker and the second location of the second marker.
projecting the known distance between markers on each of the perceived location of the radiopaque instrument using the pose of the second imaging modality comparing the measured distance to each of projected distances between the two markers to identify the true instrument location inside the body.

In some embodiments, the radiopaque instrument comprises an endoscope, an endo-bronchial tool, or a robotic arm.

In some embodiments, the method further comprises: identifying a depth of the radiopaque instrument by use of a trajectory of the radiopaque instrument.

In some embodiments, the first image from the first imaging modality is a pre-operative image. In some embodiments, the at least one image of the radiopaque instrument from the second imaging modality is an intra-operative image.

In some embodiments, a method includes receiving a sequence of medical images captured by a medical imaging device while the medical imaging device is rotated through a rotation, wherein the sequence of medical images show an area of interest that includes a plurality of landmarks; determining a pose of each of a subset of the sequence of medical images in which the plurality of landmarks are visible; estimating a trajectory of movement of the medical imaging device based on the determined poses of the subset of the sequence of medical images and a trajectory constraint of the imaging device; determining a pose of at least one of the medical images in which the plurality of landmarks are at least partially not visible by extrapolating based on an assumption of continuity of movement of the medical imaging device; and determining a volumetric reconstruction for the area of interest based at least on (a) at least some of the poses of the subset of the sequence of medical images in which the plurality of landmarks are visible and (b) at least one of the poses of the at least one of the medical images in which the plurality of landmarks are at least partially not visible.

In some embodiments, the poses of each of the subset of the sequence of medical images are determined based on 2D-3D correspondences between 3D positions of the plurality of landmarks and 2D positions of the plurality of landmarks as viewed in the subset of the sequence of medical images. In some embodiments, the 3D positions of the plurality of landmarks are determined based on at least one preoperative image. In some embodiments, the 3D positions of the plurality of landmarks are determined by application of a structure from motion technique.

In some embodiments, a method includes receiving a plurality of medical images using an imaging device mounted to a C-arm while the medical imaging device is rotated through a motion of the C-arm having a constrained trajectory, wherein at least some of the plurality of medical images include an area of interest; determining a pose of each of a subset of the plurality of medical images; calculating locations of a plurality of 3D landmarks based on 2D locations of the 3D landmarks in the subset of the plurality of medical images and based on the determined poses of each of the subset of the plurality of medical images; determining a pose of a further one of the plurality of medical images in which at least some of the 3D landmarks are visible by determining an imaging device position and an imaging device orientation based at least on a known 3D-2D correspondence of the 3D landmark; and calculating a volumetric reconstruction of the area of interest based on at least the further one of the plurality of medical images and the pose of the further one of the plurality of medical images.

In some embodiments, the pose of each of the subset of the plurality of medical images is determined based at least on a pattern of radiopaque markers visible in the subset of the plurality of medical images. In some embodiments, the pose is further determined based on the constrained trajectory.

In some embodiments, a method includes receiving a sequence of medical images captured by a medical imaging device while the medical imaging device is rotated through a rotation, wherein the sequence of medical images show an area of interest including a landmark having a 3D shape; calculating a pose of each of at least some of the medical images based on at least 3D-2D correspondence of a 2D projection of the landmark in each of the at least some of the medical images; and calculating a volumetric reconstruction of the area of interest based on at least the at least some of the medical images and the calculated poses of the at least some of the medical images.

In some embodiments, the landmark is an anatomical landmark. In some embodiments, the 3D shape of the anatomical landmark is determined based at least on at least one preoperative image.

In some embodiments, the 3D shape of the landmark is determined based at least on applying a structure from motion technique to at least some of the sequence of medical images. In some embodiments, the structure from motion technique is applied to all of the sequence of medical images.

In some embodiments, the pose is calculated for all of the sequence of medical images.

In some embodiments, the sequence of images does not show a plurality of radiopaque markers.

In some embodiments, the calculating a pose of each of the at least some of the medical images is further based on a known trajectory of the rotation.

In some embodiments, the 3D shape of the landmark is determined based on at least one preoperative image and further based on applying a structure from motion technique to at least some of the sequence of medical images.

In some embodiments, the landmark is an instrument positioned within a body of a patient at the area of interest.

In some embodiments, the landmark is an object positioned proximate to a body of a patient and outside the body of the patient. In some embodiments, the object is fixed to the body of the patient.

### BRIEF DESCRIPTION OF THE FIGURES

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

FIG. 1 shows a block diagram of a multi-view pose estimation method used in some embodiments of the method of the present invention.

FIGS. 2, 3, and 4 show an exemplary embodiments of intraoperative images used in the method of the present invention. FIGS. 2 and 3 illustrate a fluoroscopic image obtained from one specific pose. FIG. 4 illustrates a fluoroscopic image obtained in a different pose, as compared to FIGS. 2 and 3, as a result of C-arm rotation. The Bronchoscope – 240, 340, 440, the instrument – 210, 310, 410, ribs – 220, 320, 420 and body boundary - 230, 330, 430 are visible. The multi view pose estimation method uses the visible elements in FIGS. 2, 3, 4 as an input.

FIG. 5 shows a schematic drawing of the structure of bronchial airways as utilized in the method of the present invention. The airways centerlines are represented by 530. A catheter is inserted into the airways structure and imaged by a fluoroscopic device with an image plane 540. The catheter projection on the image is illustrated by the curve 550 and the radio opaque markers attached to it are projected into points G and F.

FIG. 6 is an image of a bronchoscopic device tip attached to a bronchoscope, in which the bronchoscope can be used in an embodiment of the method of the present invention.

FIG. 7 is an illustration according to an embodiment of the method of the present invention, where the illustration is of a fluoroscopic image of a tracked scope (701) used in a bronchoscopic procedure with an operational tool (702) that extends from it. The operational tool may contain radio opaque markers or unique pattern attached to it.

FIG. 8 is an illustration of epipolar geometry of two views according to an embodiment of the method of the present invention, where the illustration is of a pair of fluoroscopic images containing a scope (801) used in a bronchoscopic procedure with an operational tool (802) that extends from it. The operational tool may contain radio opaque markers or unique pattern attached to it (points P1 and P2 are representing a portion of such pattern). The point P1 has a corresponding epipolar line L1. The point P0 represents the tip of the scope and the point P3 represents the tip of the operational tool. O1 and O2 denote the focal points of the corresponding views.

FIG. 9 shows an exemplary method for 6-degree-of-freedom pose estimation from 3D-2D correspondences.

FIGS. 10A and 10B show poses of an X-ray imaging device mounted on a C-arm.

FIG. 11 shows use of 3D landmarks use to estimate trajectory of a C-arm.

FIG. 12 shows a method for an algorithm to use a visible and known set of radiopaque markers to estimate a pose per each image frame.

FIG. 13 shows a method for estimating 3D landmarks using a structure from motion approach without use of radiopaque markers.

FIG. 14 shows a same feature point of an object visible in multiple frames.

FIG. 15 shows a same feature point of an object visible in multiple frames.

FIG. 16 shows a method for optimizing determination of location of a feature point of an object visible in multiple frames.

FIG. 17 shows a process for determining a 3D image reconstruction based on a received sequence of 2D images.

FIG. 18 shows a process for training an image-to-image translation using unaligned images.

FIG. 19 shows training for a model for translation from domain C to domain B.

FIG. 20 shows exemplary guidance for a user to position a fluoroscope.

FIG. 21 shows exemplary guidance for a user to position a fluoroscope.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

### DETAILED DESCRIPTION

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiments, though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, a “plurality” refers to more than one in number, e.g., but not limited to, 2, 3, 4, 5, 6, 7, 8, 9, 10, etc. For example, a plurality of images can be 2 images, 3 images, 4 images, 5 images, 6 images, 7 images, 8 images, 9 images, 10 images, etc.

As used herein, an “anatomical element” refers to a landmark, which can be, e.g.: an area of interest, an incision point, a bifurcation, a blood vessel, a bronchial airway, a rib or an organ.

As used herein, “geometrical constraints” or “geometric constraints” or “mutual constraints” or “mutual geometric constraints” refer to a geometrical relationship between physical organs (e.g., at least two physical organs) in a subject’s body which construct a similar geometric relationship within the subject between ribs, the boundary of the body, etc. Such geometrical relationships, as being observed through different imaging modalities, either remain unchanged or their relative movement can be neglected or quantified.

As used herein, a “pose” refers to a set of six parameters that determine a relative position and orientation of the intraoperative imaging device source as a substitute to the optical camera device. As a non-limiting example, a pose can be obtained as a combination of relative movements between the device, patient bed, and the patient. Another non-limiting example of such movement is the rotation of the intraoperative imaging device combined with its movement around the static patient bed with static patient on the bed.

As used herein, a “position” refers to the location (that can be measured in any coordinate system such as x, y, and z Cartesian coordinates) of any object, including an imaging device itself within a 3D space.

As used herein, an “orientation” refers the angles of the intraoperative imaging device. As non-limiting examples, the intraoperative imaging device can be oriented facing upwards, downwards, or laterally.

As used herein, a “pose estimation method” refers to a method to estimate the parameters of a camera associated with a second imaging modality within the 3D space of the first imaging modality. A non-limiting example of such a method is to obtain the parameters of the intraoperative fluoroscopic camera within the 3D space of a preoperative CT. A mathematical model uses such estimated pose to project at least one 3D point inside of a preoperative computed tomography (CT) image to a corresponding 2D point inside the intraoperative X-ray image.

As used herein, a “multi view pose estimation method” refers a method to estimate to poses of at least two different poses of the intraoperative imaging device. Where the imaging device acquires image from the same scene/subject.

As used herein, “relative angular difference” refers to the angular difference of the between two poses of the imaging device caused by their relative angular movement.

As used herein, “relative pose difference” refers to both location and relative angular difference between two poses of the imaging device caused by the relative spatial movement between the subject and the imaging device.

As used herein, “epipolar distance” refers to a measurement of the distance between a point and the epipolar line of the same point in another view. As used herein, an “epipolar line” refers to a calculation from an x, y vector or two-column matrix of a point or points in a view.

As used herein, a “similarity measure” refers to a real-valued function that quantifies the similarity between two objects.

In some embodiments, the present invention provides a method, comprising:
obtaining a first image from a first imaging modality,
extracting at least one element from the first image from the first imaging modality, wherein the at least one element comprises an airway, a blood vessel, a body cavity, or any combination thereof;
obtaining, from a second imaging modality, at least (i) a first image of a radiopaque instrument in a first pose and (ii) a second image of the radiopaque instrument in a second pose,
   wherein the radiopaque instrument is in a body cavity of a patient; generating at least two augmented bronchograms,
   wherein a first augmented bronchogram corresponds to the first image of the radiopaque instrument in the first pose, and
   wherein a second augmented bronchogram corresponds to the second image of the radiopaque instrument in the second pose,
determining mutual geometric constraints between:
   (i) the first pose of the radiopaque instrument, and
   (ii) the second pose of the radiopaque instrument,
estimating the first pose of the radiopaque instrument and the second pose of the radiopaque instrument by comparing the first pose of the radiopaque instrument and the second pose of the radiopaque instrument to the first image of the first imaging modality,
   wherein the comparing is performed using:
      (i) the first augmented bronchogram,
      (ii) the second augmented bronchogram, and
      (iii) the at least one element, and
   wherein the estimated first pose of the radiopaque instrument and the estimated second pose of the radiopaque instrument meets the determined mutual geometric constraints,
generating a third image; wherein the third image is an augmented image derived from the second imaging modality which highlights an area of interest,
wherein the area of interest is determined from data from the first imaging modality.

In some embodiments, the at least one element from the first image from the first imaging modality further comprises a rib, a vertebra, a diaphragm, or any combination thereof. In some embodiments, the mutual geometric constraints are generated by:
a. estimating a difference between (i) the first pose and (ii) the second pose by comparing the first image of the radiopaque instrument and the second image of the radiopaque instrument,
   wherein the estimating is performed using a device comprising a protractor, an accelerometer, a gyroscope, or any combination thereof, and wherein the device is attached to the second imaging modality;
b. extracting a plurality of image features to estimate a relative pose change,
   wherein the plurality of image features comprise anatomical elements, non-anatomical elements, or any combination thereof,
   wherein the image features comprise: patches attached to a patient, radiopaque markers positioned in a field of view of the second imaging modality, or any combination thereof,
   wherein the image features are visible on the first image of the radiopaque instrument and the second image of the radiopaque instrument;
c. estimating a difference between (i) the first pose and (ii) the second pose by using a at least one camera,
   wherein the camera comprises: a video camera, an infrared camera, a depth camera, or any combination thereof,
   wherein the camera is at a fixed location,
   wherein the camera is configured to track at least one feature,
      wherein the at least one feature comprises: a marker attached the patient, a marker attached to the second imaging modality, or any combination thereof, and
      tracking the at least one feature;
d. or any combination thereof.

In some embodiments, the method further comprises: tracking the radiopaque instrument for: identifying a trajectory, and using the trajectory as a further geometric constraint, wherein the radiopaque instrument comprises an endoscope, an endo-bronchial tool, or a robotic arm.

In some embodiments, the present invention is a method, comprising:
generating a map of at least one body cavity of the patient,
   wherein the map is generated using a first image from a first imaging modality, obtaining, from a second imaging modality, an image of a radiopaque instrument comprising at least two attached markers,
   wherein the at least two attached markers are separated by a known distance, identifying a pose of the radiopaque instrument from the second imaging modality relative to a map of at least one body cavity of a patient,
identifying a first location of the first marker attached to the radiopaque instrument on the second image from the second imaging modality,
identifying a second location of the second marker attached to the radiopaque instrument on the second image from the second imaging modality, and
measuring a distance between the first location of the first marker and the second location of the second marker,
projecting the known distance between the first marker and the second marker, comparing the measured distance with the projected known distance between the first marker and the second marker to identify a specific location of the radiopaque instrument inside the at least one body cavity of the patient.
It is possible that inferred 3d information from a single view is still ambiguous and can fit the tool into multiple locations inside the lungs. The occurrence of such situations can be reduced by analyzing the planned 3d path before the actual procedure and calculating the most optimal orientation of the fluoroscope to avoid the majority of ambiguities during the navigation.In some embodiments, the fluoroscope positioning is performed in accordance with the methods described in the claim 4 of the International Patent Application No. PCT/IB2015/00438, the contents of which are incorporated herein by reference in their entirety.

In some embodiments, the radiopaque instrument comprises an endoscope, an endo-bronchial tool, or a robotic arm.

In some embodiments, the method further comprises: identifying a depth of the radiopaque instrument by use of a trajectory of the radiopaque instrument.

In some embodiments, the first image from the first imaging modality is a pre-operative image. In some embodiments, the at least one image of the radiopaque instrument from the second imaging modality is an intra-operative image.

In some embodiments, the present invention is a method, comprising:
obtaining a first image from a first imaging modality,
extracting at least one element from the first image from the first imaging modality,
   wherein the at least one element comprises an airway, a blood vessel, a body cavity or any combination thereof;
obtaining, from a second imaging modality, at least (i) a one image of a radiopaque instrument and (ii) another image of the radiopaque instrument in two different poses of second imaging modality
   wherein the first image of the radiopaque instrument is captured at a first pose of second imaging modality,
   wherein the second image of the radiopaque instrument is captured at a second pose of second imaging modality, and
   wherein the radiopaque instrument is in a body cavity of a patient;
generating at least two augmented bronchograms correspondent to each of two poses of the imaging device, wherein a first augmented bronchogram derived from the first image of the radiopaque instrument and the second augmented bronchogram derived from the second image of the radiopaque instrument,
determining mutual geometric constraints between:
   (i) the first pose of the second imaging modality, and
   (ii) the second pose of the second imaging modality,
estimating the two poses of the second imaging modality relatively to the first image of the first imaging modality, using the correspondent augmented bronchogram images and at least one element extracted from the first image of the first imaging modality;
   wherein the two estimated poses satisfy the mutual geometric constrains.
   generating a third image; wherein the third image is an augmented image derived from the second imaging modality highlighting the area of interest, based on data sourced from the first imaging modality.

During navigation of the endobronchial tool there is a need to verify tool location in 3D relatively to the target and other anatomical structures. After reaching some location in the lungs a physician may change the fluoroscope position while keeping the tool at the same location. Using these intraoperative images skilled in the art can reconstruct the tool position in 3d and show the physician the tool position in relation to the target in 3d.

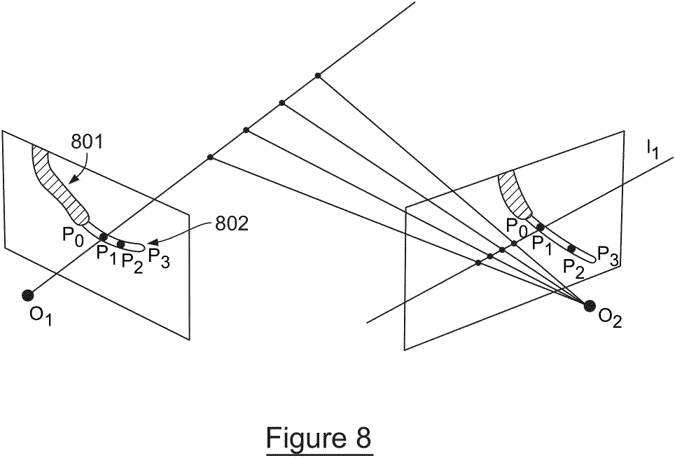

In order to reconstruct the tool position in 3d it is required to pick the corresponding points on both views. The points can be special markers on the tool, or identifiable points on any instrument, for example, a tip of the tool, or a tip of the bronchoscope. To achieve this, epipolar lines can be used to find the correspondence between points. In addition, epipolar constraints can be used to filter false positive marker detections and also to exclude markers that don’t have a corresponding pair due to marker miss-detection (see FIG. 8).

(Epipolar is related to the geometry of the stereo vision, special area of computational geometry)

In some embodiments, the virtual markers can be generated on any instrument, for instance instruments not having visible radiopaque markers. It is performed by: (1) selecting any point on the instrument on the first image (2) calculating epipolar line on the second image using known geometric relation between both images; (3) intersecting epipolar lines with the known or instrument trajectory from the second image, giving a matching virtual marker.

In some embodiments, the present invention is a method, comprising:
obtaining a first image from a first imaging modality,
extracting at least one element from the first image from the first imaging modality,
   wherein the at least one element comprises an airway, a blood vessel, a body cavity or any combination thereof;
obtaining, from a second imaging modality, at least two images in two different poses of second imaging modality of the same radiopaque instrument position for at least one or more different instrument positions,
   wherein the radiopaque instrument is in a body cavity of a patient;
reconstructing the 3D trajectory of each instrument from the corresponding multiple images of the same instrument position in the reference coordinate system, using mutual geometric constraints between poses of the corresponding images;
estimating transformation between the reference coordinate system and the image of the first imaging modality by estimating the transform that fits reconstructed 3D trajectories of positions of radiopaque instrument with the 3D trajectories extracted from the image of the first imaging modality;
generating a third image; wherein the third image is an augmented image derived from the second imaging modality with the known pose in a reference coordinate system and highlighting the area of interest, based on data sourced from the first imaging modality using the transformation between the reference coordinate system and the image of the first imaging modality.

In some embodiments, a method of collecting the images from different poses of the multiple radiopaque instrument positions, is comprising of: (1) positioning a radiopaque instrument in the first position; (2) taking an image of the second imaging modality; (3) change a pose of the second modality imaging device; (4) taking another image of the second imaging modality; (5) changing the radiopaque instrument position; (6) proceeding with step 2, until the desired number of unique radiopaque instrument positions is achieved.

In some embodiments, it is possible to reconstruct the location of any element that can be identified on at least two intraoperative images originated from two different poses of the imaging device. When each pose of the second imaging modality relatively to the first image of the first imaging modality is known, it is possible to show the element’s reconstructed 3D position with respect to any anatomical structure from the image of the first imaging modality. As an example of the usage of this technique can be a confirmation of 3D positions of the deployed fiducial markers relatively to the target.

In some embodiments, the present invention is a method, comprising:
obtaining a first image from a first imaging modality,
extracting at least one element from the first image from the first imaging modality,
   wherein the at least one element comprises an airway, a blood vessel, a body cavity or any combination thereof;
obtaining, from a second imaging modality, at least (i) a one image of a radiopaque fiducials and (ii) another image of the radiopaque fiducials in two different poses of second imaging modality
   wherein the first image of the radiopaque fiducials is captured at a first pose of second imaging modality,
   wherein the second image of the radiopaque fiducials is captured at a second pose of second imaging modality;
reconstructing the 3D position of radiopaque fiducials from two poses of the imaging device, using mutual geometric constraints between:
   (i) the first pose of the second imaging modality, and
   (ii) the second pose of the second imaging modality,
generating a third image showing the relative 3D position of the fiducials relatively to the area of interest, based on data sourced from the first imaging modality.

In some embodiments, anatomical elements such as: a rib, a vertebra, a diaphragm, or any combination thereof, are extracted from the first imaging modality and from the second imaging modality.

In some embodiments, the mutual geometric constraints are generated by:
a. estimating a difference between (i) the first pose and (ii) the second pose by comparing the first image of the radiopaque instrument and the second image of the radiopaque instrument,
   wherein the estimating is performed using a device comprising a protractor, an accelerometer, a gyroscope, or any combination thereof, and
   wherein the device is attached to the second imaging modality;
b. extracting a plurality of image features to estimate a relative pose change,
   wherein the plurality of image features comprise anatomical elements, non-anatomical elements, or any combination thereof,
   wherein the image features comprise: patches attached to a patient, radiopaque markers positioned in a field of view of the second imaging modality, or any combination thereof,
   wherein the image features are visible on the first image of the radiopaque instrument and the second image of the radiopaque instrument;
c. estimate a difference between (i) the first pose and (ii) the second pose by using a at least one camera,
   wherein the camera comprises: a video camera, an infrared camera, a depth camera, or any combination thereof,
   wherein the camera is at a fixed location,
   wherein the camera is configured to track at least one feature,
      wherein the at least one feature comprises: a marker attached the patient, a marker attached to the second imaging modality, or any combination thereof, and
   tracking the at least one feature;
d. or any combination thereof.

In some embodiments, the method further comprises tracking the radiopaque instrument to identify a trajectory and using such trajectory as additional geometric constrains, wherein the radiopaque instrument comprises an endoscope, an endo-bronchial tool, or a robotic arm.

In some embodiments, the present invention is a method to identify the true instrument location inside the patient, comprising:
using a map of at least one body cavity of a patient generated from a first image of a first imaging modality,
obtaining, from a second imaging modality, an image of the radiopaque instrument with at least two markers attached to it and having the defined distance between them ,
that may be perceived from the image as located in at least two different body cavities inside the patient,
obtaining the pose of the second imaging modality relative to the map
identifying a first location of the first marker attached to the radiopaque instrument on the second image from the second imaging modality,
identifying a second location of the second marker attached to the radiopaque instrument on the second image from the second imaging modality, and
measuring a distance between the first location of the first marker and the second location of the second marker.
projecting the known distance between markers on each of the perceived location of the radiopaque instrument using the pose of the second imaging modality
comparing the measured distance to each of projected distances between the two markers to identify the true instrument location inside the body.

In some embodiments, the radiopaque instrument comprises an endoscope, an endo-bronchial tool, or a robotic arm.

In some embodiments, the method further comprises: identifying a depth of the radiopaque instrument by use of a trajectory of the radiopaque instrument.

In some embodiments, the first image from the first imaging modality is a pre-operative image. In some embodiments, the at least one image of the radiopaque instrument from the second imaging modality is an intra-operative image.

### Multi View Pose Estimation

The application PCT/IB2015/000438 includes a description of a method to estimate the pose information (e.g., position, orientation) of a fluoroscope device relative to a patient during an endoscopic procedure, and is herein incorporated by reference in its entirety. PCT/IB 15/002148 filed Oct. 20, 2015 is also herein incorporated by reference in its entirety.

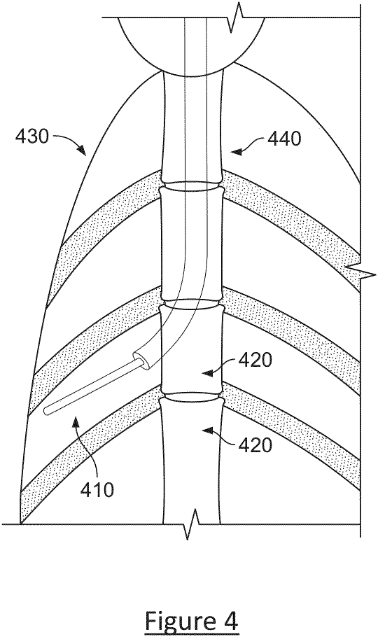

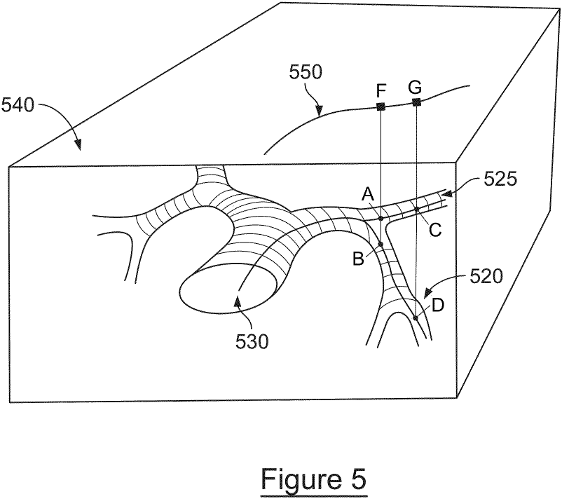

The present invention is a method which includes data extracted from a set of intra-operative images, where each of the images is acquired in at least one (e.g., 1, 2, 3, 4, etc.) unknown pose obtained from an imaging device. These images are used as input for the pose estimation method. As an exemplary embodiment, FIGS. 3, 4, 5, are examples of a set of 3 Fluoroscopic images. The images in FIGS. 4 and 5 were acquired in the same unknown pose while the image in FIG. 3 was acquired in a different unknown pose. This set, for example, may or may not contain additional known positional data related to the imaging device. For example, a set may contain positional data, such as C-arm location and orientation, which can be provided by a Fluoroscope or acquired through a measurement device attached to the Fluoroscope, such as protractor, accelerometer, gyroscope, etc.

In some embodiments, anatomical elements are extracted from additional intraoperative images and these anatomical elements imply geometrical constraints which can be introduced into the pose estimation method. As a result, the number of elements extracted from a single intraoperative image can be reduced prior to using the pose estimation method.

In some embodiments, the multi view pose estimation method further includes overlaying information sourced from a pre-operative modality over any image from the set of intraoperative images.

In some embodiments, a description of overlaying information sourced from a pre-operative modality over intraoperative images can be found in PCT/IB2015/000438,which is incorporated herein by reference in its entirety.

In some embodiments, the plurality of second imaging modalities allow for changing a Fluoroscope pose relatively to the patient (e.g., but not limited to, a rotation or linear movement of the Fluoroscope arm, patient bed rotation and movement, patient relative movement on the bed, or any combination of the above) to obtain the plurality of images, where the plurality of images are obtained from abovementioned relative poses of the fluoroscopic source as any combination of rotational and linear movement between the patient and Fluoroscopic device.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

Reference is now made to the following examples, which together with the above descriptions illustrate some embodiments of the invention in a non limiting fashion.

### Example: Minimally Invasive Pulmonary Procedure

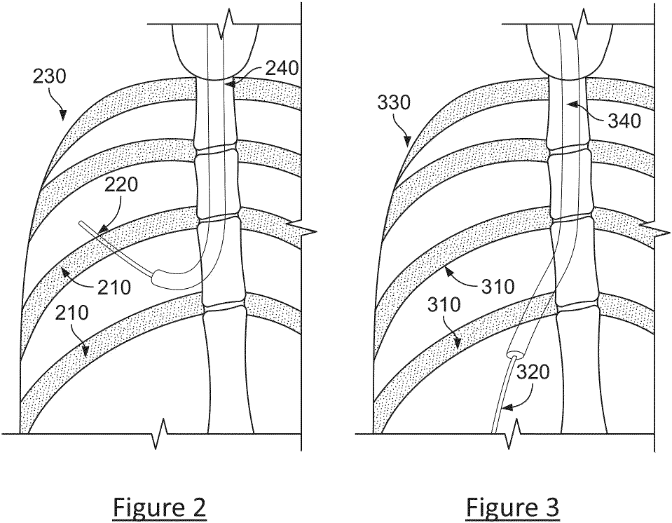

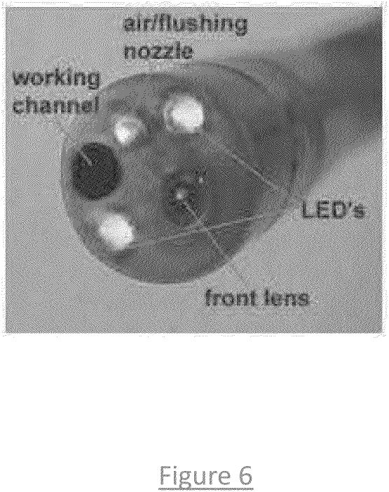

A non-limiting exemplary embodiment of the present invention can be applied to a minimally invasive pulmonary procedure, where endo-bronchial tools are inserted into bronchial airways of a patient through a working channel of the Bronchoscope (see FIG. 6). Prior to commencing a diagnostic procedure, the physician performs a Setup process, where the physician places a catheter into several (e.g., 2, 3, 4, etc.) bronchial airways around an area of interest. The Fluoroscopic images are acquired for every location of the endo-bronchial catheter, as shown in FIGS. 2, 3, and 4. An example of the navigation system used to perform the pose estimation of the intra-operative Fluoroscopic device is described in application PCT/IB2015/000438, and the present method of the invention uses the extracted elements (e.g., but not limited to, multiple catheter locations, rib anatomy, and a patient’s body boundary).

After estimating the pose in the area of interest, pathways for inserting the bronchoscope can be identified on a pre-procedure imaging modality, and can be marked by highlighting or overlaying information from a pre-operative image over the intraoperative Fluoroscopic image. After navigating the endo-bronchial catheter to the area of interest, the physician can rotate, change the zoom level, or shift the Fluoroscopic device for, e.g., verifying that the catheter is located in the area of interest. Typically, such pose changes of the Fluoroscopic device, as illustrated by FIG. 4, would invalidate the previously estimated pose and require that the physician repeats the Setup process. However, since the catheter is already located inside the potential area of interest, repeating the Setup process need not be performed.

FIG. 4 shows an exemplary embodiment of the present invention, showing the pose of the Fluoroscope angle being estimated using anatomical elements, which were extracted from FIGS. 2 and 3 (in which, e.g., FIGS. 2 and 3 show images obtained from the initial Setup process and the additional anatomical elements extracted from image, such as catheter location, ribs anatomy and body boundary). The pose can be changed by, for example, (1) moving the Fluoroscope (e.g., rotating the head around the c-arm), (2) moving the Fluoroscope forward are backwards, or alternatively through the subject position change or either through the combination of both etc. In addition, the mutual geometric constraints between FIG. 2 and FIG. 4, such as positional data related to the imaging device, can be used in the estimation process.

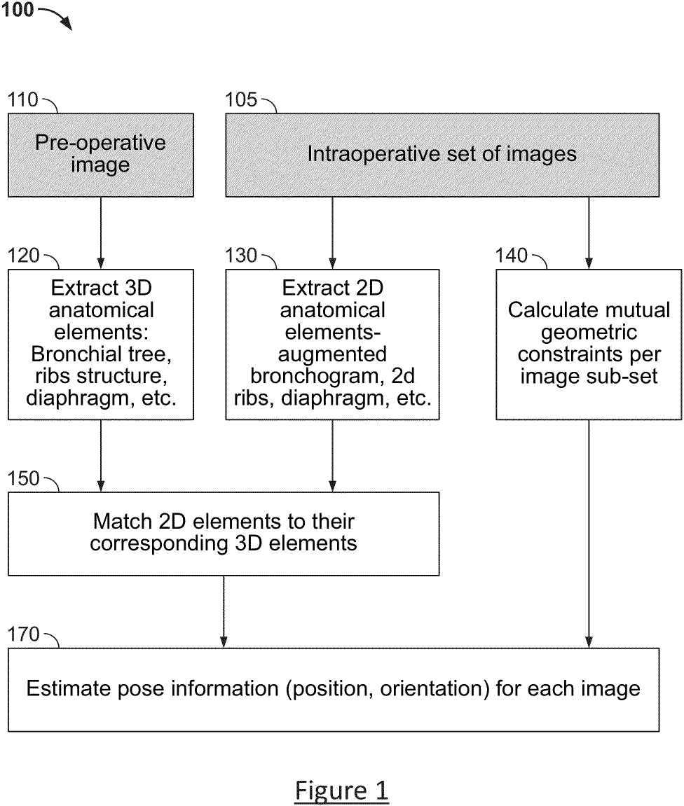

FIG. 1 is an exemplary embodiment of the present invention, and shows the following:

I. The component 120 extracts 3D anatomical elements, such as Bronchial airways, ribs, diaphragm, from the preoperative image, such as, but not limited to, CT, magnetic resonance imaging (MRI), Positron emission tomography-computed tomography (PET-CT), using an automatic or semi-automatic segmentation process, or any combination thereof. Examples of automatic or semi-automatic segmentation processes are described in “Three-dimensional Human Airway Segmentation Methods for Clinical Virtual Bronchoscopy”, Atilla P. Kiraly, William E. Higgins, Geoffrey McLennan, Eric A. Hoffman, Joseph M. Reinhardt, which is hereby incorporated by reference in its entirety.

II. The component 130 extracts 2D anatomical elements (which are further shown in FIG. 4, such as Bronchial airways 410, ribs 420, body boundary 430 and diaphragm) from a set of intraoperative images, such as, but not limited to, Fluoroscopic images, ultrasound images, etc.

III. The component 140 calculates the mutual constraints between each subset of the images in the set of intraoperative images, such as relative angular difference, relative pose difference, epipolar distance, etc.

In another embodiment, the method includes estimating the mutual constraints between each subset of the images in the set of intraoperative images. Non-limiting examples of such methods are: (1) the use of a measurement device attached to the intraoperative imaging device to estimate a relative pose change between at least two poses of a pair of fluoroscopic images. (2) The extraction of image features, such as anatomical elements or non-anatomical elements including, but not limited to, patches (e.g., ECG patches) attached to a patient or radiopaque markers positioned inside the field of view of the intraoperative imaging device, that are visible on both images, and using these features to estimate the relative pose change. (3) The use of a set of cameras, such as video camera, infrared camera, depth camera, or any combination of those, attached to the specified location in the procedure room, that tracks features, such as patches attached to the patient or markers, markers attached to imaging device, etc. By tracking such features the component can estimate the imaging device relative pose change.

IV. The component 150 matches the 3D element generated from preoperative image to their corresponding 2D elements generated from intraoperative image. For example, matching a given 2D Bronchial airway extracted from Fluoroscopic image to the set of 3D airways extracted from the CT image.

V. The component 170 estimates the poses for the each of the images in the set of intra-operative images in the desired coordinate system, such as preoperative image coordinate system, operation environment related, coordinated system formed by other imaging or navigation device, etc.

The inputs to this component are as follows:
3D anatomical elements extracted from the patient preoperative image.
2D anatomical elements extracted from the set of intra-operative images. As stated herein, the images in the set can be sourced from the same or different imaging device poses.
Mutual constraints between each subset of the images in the set of intraoperative images

The component 170 evaluates the pose for each image from the set of intra-operative images such that:
The 2D extracted elements match the correspondent and projected 3D anatomical elements.
The mutual constraint conditions calculated by the component 140 apply for the estimated poses.

To match the projected 3D elements, sourcing a preoperative image to the correspondent 2D elements from an inter-operative image, a similarity measure, such as a distance metric, is needed. Such a distance metric provides a measure to assess the distances between the projected 3D elements and their correspondent 2D elements. For example, a Euclidian distance between 2 polylines (e.g., connected sequence of line segments created as a single object) can be used as a similarity measure between 3D projected Bronchial airway sourcing pre-operative image to 2D airway extracted from the intra-operative image.

Additionally, in an embodiment of the method of the present invention, the method includes estimating a set of poses that correspond to a set of intraoperative images by identifying such poses which optimize a similarity measure, provided that the mutual constraints between the subset of images from intraoperative image set are satisfied. The optimization of the similarity measure can be referred to as a Least Squares problem and can be solved in several methods, e.g., (1) using the well-known bundle adjustment algorithm which implements an iterative minimization method for pose estimation, and which is herein incorporated by reference in its entirety: B. Triggs; P. McLauchlan; R. Hartley; A. Fitzgibbon (1999) “Bundle Adjustment ------A Modern Synthesis”. ICCV ‘99: Proceedings of the International Workshop on Vision Algorithms. Springer-Verlag. pp. 298-372, and (2) using a grid search method to scan the parameter space in search for optimal poses that optimize the similarity measure.

### Markers

Radio-opaque markers can be placed in predefined locations on the medical instrument in order to recover 3D information about the instrument position. Several pathways of 3D structures of intra-body cavities, such as bronchial airways or blood vessels, can be projected into similar 2D curves on the intraoperative image. The 3D information obtained with the markers may be used to differentiate between such pathways, as shown, e.g., in Application PCT/IB2015/000438.

In an exemplary embodiment of the present invention, as illustrated by FIG. 5, an instrument is imaged by an intraoperative device and projected to the imaging plane 505. It is unknown whether the instrument is placed inside airway 520 or airway 525 since both airways are projected into the same curve on the imaging plane 505. In order to differentiate between airway 520 and airway 525, it is possible to use at least 2 radiopaque markers attached to the catheter having predefined distance “m” between the markers. In FIG. 5, the markers observed on the preoperative image are named “G” and “F”.

The differentiation process between airway 520 and airway 525 can be performed as follows:
(1) Project point F from intraoperative image on the potential candidates of correspondent airways 520, 525 to obtain A and B points.
(2) Project point G from intraoperative image on the potential candidates of correspondent airways 520, 525 to obtain points C and D.
(3) Measure the distance between pairs of projected markers |AC| and |BD|.
(4) Compare the distances |AC| on 520 and |BD| on 525 to the distance m predefined by tool manufacturer. Choose appropriate airway according to a distance similarity.

### Tracked Scope

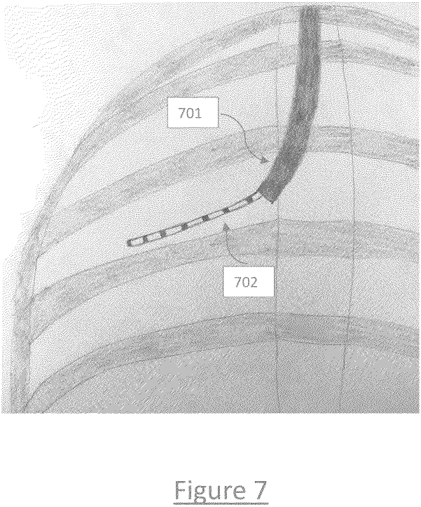

As non-limiting examples, methods to register a patient CT scan with a Fluoroscopic device are disclosed herein. This method uses anatomical elements detected both in the Fluoroscopic image and in the CT scan as an input to a pose estimation algorithm that produces a Fluoroscopic device Pose (e.g., orientation and position) with respect to the CT scan. The following extends this method by adding 3D space trajectories, corresponding to an endo-bronchial device position, to the inputs of the registration method. These trajectories can be acquired by several means, such as: attaching positional sensors along a scope or by using a robotic endoscopic arm. Such an endo-bronchial device will be referred from now on as Tracked Scope. The Tracked scope is used to guide operational tools that extends from it to the target area (see FIG. 7). The diagnostic tools may be a catheter, forceps, needle, etc. The following describes how to use positional measurements acquired by the Tracked scope to improve the accuracy and robustness of the registration method shown herein.

In one embodiment, the registration between Tracked Scope trajectories and coordinate system of Fluoroscopic device is achieved through positioning of the Tracked Scope in various locations in space and applying a standard pose estimation algorithm. See the following paper for a reference to a pose estimation algorithm: F. Moreno-Noguer, V. Lepetit and P. Fua in the paper “EPnP: Efficient Perspective-n-Point Camera Pose Estimation”, which is hereby incorporated by reference in its entirety.

The pose estimation method disclosed herein is performed through estimating a Pose in such way that selected elements in the CT scan are projected on their corresponding elements in the fluoroscopic image. In one embodiment of the present invention, adding the Tracked Scope trajectories as an input to the pose estimation method extends this method. These trajectories can be transformed into the Fluoroscopic device coordinate system using the methods herein. Once transformed to the Fluoroscopic device coordinate system, the trajectories serve as additional constraints to the pose estimation method, since the estimated pose is constrained by the condition that the trajectories must fit the bronchial airways segmented from the registered CT scan.

The Fluoroscopic device estimated Pose may be used to project anatomical elements from the pre-operative CT to the Fluoroscopic live video in order to guide an operational tool to a specified target inside the lung. Such anatomical elements may be, but are not limited to,: a target lesion, a pathway to the lesion, etc. The projected pathway to the target lesion provides the physician with only two-dimensional information, resulting in a depth ambiguity, that is to say several airways segmented on CT may correspond to the same projection on the 2D Fluoroscopic image. It is important to correctly identify the bronchial airway on CT in which the operational tool is placed. One method used to reduce such ambiguity, described herein, is performed by using radiopaque markers placed on the tool providing depth information. In another embodiment of the present invention, the Tracked scope may be used to reduce such ambiguity since it provides the 3D position inside the bronchial airways. Having such approach applied to the brunching bronchial tree, it allows eliminating the potential ambiguity options until the Tracked Scope tip 701 on FIG. 7. Assuming the operational tool 702 on FIG. 7 does not have the 3D trajectory, although the abovementioned ambiguity may still happen for this portion of the tool 702, such event is much less probable to occur. Therefore this embodiment of present invention improves the ability of the method described herein to correctly identify the present tool’s position.

### Digital Computational Tomography (DCT)

In some embodiments, the tomography reconstruction from intraoperative images can be used for calculating the target position relatively to the reference coordinate system. A non-limiting example of such a reference coordinate system can be defined by a jig with radiopaque markers with known geometry, allowing to calculate a relative pose of each intraoperative image. Since each input frame of the tomographic reconstructions has known geometric relationship to a reference coordinate system, the position of the target is also can be positioned in the reference coordinate system. This allows to project a target on further fluoroscopic images. In some embodiments, the projected target position can be compensated for respiratory movement by tracking tissue in the region of the target. In some embodiments, the movement compensation is performed in accordance with the exemplary methods described in International Patent Application No. PCT/IB2015/00438, the contents of which are incorporated herein by reference in their entirety.

A method for augmenting target on intraoperative images using the C-arm based CT and reference pose device, comprising of:
collecting multiple intraoperative images with known geometric relation to a reference coordinate system;
   reconstructing 3d volume;
   marking the target area on the reconstructed volume;
   projecting target on further intraoperative images with known geometric relation to a reference coordinate system.

In other embodiments, the tomography reconstructed volume can be registered to the preoperative CT volume. Given the known position of the center of the target, or anatomical structures adjunctive to the target, such as blood vessels, bronchial airways, or airway bifurcations in the reconstructed volume and in the preoperative volume, both volumes can be initially aligned. In other embodiments, ribs extracted from both volumes can be used to find an alignment (e.g., an initial alignment). In some embodiments, a step of finding the correct rotation between the volumes the reconstructed position and trajectory of the instrument can be matched to all possible airway trajectories extracted from the CT. The best match will define the most optimal relative rotation between the volumes.

In some embodiments, the tomography reconstructed volume can be registered to the preoperative CT volume using at least 3 common anatomical landmarks that can be identified on both the tomography reconstructed volume and the preoperative CT volume. Examples of such anatomical landmarks can be airways bifurcations, blood vessels.

In some embodiments, the tomography reconstructed volume can be registered to the preoperative CT volume using image-based similarity methods such as mutual information.

In some embodiments, the tomography reconstructed volume can be registered to the preoperative CT volume using a combination of at least one common anatomical landmark (e.g., a 3D-to-3D constraint) between the tomography reconstructed volume and the preoperative CT volume and also at least one 3D-to-2D constraint (e.g., ribs or a rib cage boundary). In such embodiments, both type of constraints can be formulated as an energy function and minimized using standard optimization methods like gradient descent.

In other embodiments, the tomography reconstructed volumes from different times of the same procedure can be registered together. Some application of this could be comparison of 2 images, transferring manual markings from one image to another, showing chronological 3D information.

In other embodiments, only partial information can be reconstructed from the DCT because limited quality of fluoroscopic imaging, obstruction of the area of interest by other tissue, space limitations of the operational environment. In such cases the corresponded partial information can be identified between the partial 3d volume reconstructed from intraoperative imaging and preoperative CT. The two image sources can be fused together to form unified data set. The abovementioned dataset can be updated from time to time with additional intra procedure images.

In other embodiments, the tomography reconstructed volume can be registered to the REBUS reconstructed 3D target shape.

A method for performing CT to fluoro registration using the tomography, comprising of:
Marking a target on the preoperative image and extracting a bronchial tree;
positioning an endoscopic instrument inside the target lobe of the lungs;
performing a tomography spin using c-arm while the tool is inside and stable;
marking the target and the instrument on the reconstructed volume;
aligning the preoperative and reconstructed volumes by the target position or by position of adjunctive anatomical structures;
for all possible airway trajectories extracted from the CT, calculate
the optimal rotation between the volumes that minimizes the distance between the reconstructed trajectory of the instrument and each airway trajectory;
selecting the rotation corresponding to the minimal distance;
using the alignment between two volumes, enhancing the reconstructed volume with the anatomical information originated in the preoperative volume;
highlighting the target area on further intraoperative images.

In other embodiments, the quality of the digital tomosynthetis can be enhanced by using the prior volume of the preoperative CT scan. Given the known coarse registration between the intraoperative images and preoperative CT scan, the relevant region of interest can be extracted from the volume of the preoperative CT scan. Adding constraints to the well-known reconstruction algorithm can significantly improve the reconstructed image quality, which is herein incorporated by reference in its entirety: Sechopoulos, Ioannis (2013). “A review of breast tomosynthesis. Part II. Image reconstruction, processing and analysis, and advanced applications”. Medical Physics. 40 (1): 014302. As an example of such a constraint, the initial volume can be initialized with the extracted volume from the preoperative CT.

In some embodiments, a method of improving tomography reconstruction using the prior volume of the preoperative CT scan, comprising: performing registration between the intraoperative images and preoperative CT scan;
extracting the region of interest volume from the preoperative CT scan;
adding constraints to the well-known reconstruction algorithm;
reconstructing the image using the added constraints; and
using landmarks to estimate pose during tomography

In some embodiments, in order to perform a tomography reconstruction, multiple images of the same region from different poses are required.

In some embodiments, pose estimation can be done using fixed pattern of the 3D radiopaque markers as described in International Pat. App. No. PCT/IB 17/01448, “Jigs for use in medical imaging and methods for use thereof” (hereby incorporated by reference herein). For example, usage of such 3D patterns with radiopaque markers add a physical limitation that the said pattern has to be at least partially visible in the image frame together with the region of interest area of the patient.

For example, one such C-arm based CT system is described in the Prior Art, the U.S. Pat. Application for “C-arm computerized tomography system”, published as US 9044190B2. This application, generally uses a three-dimensional target disposing in a fixed position relative to the subject, and obtains a sequence of video images of a region of interest of a subject while the C-arm is moved manually or by a scanning motor. Images from the video sequence are analyzed to determine the pose of the C-arm relative to the subject by analysis of the image patterns of the target.

However, this system is dependent on the three-dimensional target with opaque markers that must be in the field of view for each frame in order to determine its pose. This requirement either significantly limits the imaging angles of a C-arm or, alternatively, requires positioning such three dimensional target (or the portion of the target) above or around the patient which is a limiting factor from a clinical application perspective since it is limiting the approach to the patient or the movement of the C-Arm itself. It is known that the quality and dimensionality of tomographic reconstruction among other factors depends on the C-Arm rotation angle. From the tomographic reconstruction quality perspective the C-Arm rotation angle range becomes critical for tomographic reconstruction of the small soft tissue objects. The non-limiting example representing such objects is soft-tissue lesion of 8-15 mm size inside the human lung.

Therefore there is at least a need for a system to obtain wide-angle imaging from conventional C-arm fluoroscopic imaging systems, without the need to have a limiting three dimensional target (or its portion) with opaque markers in very imaged frame in order to determine the pose of the C-Arm suitable for every such frame of the C-Arm.

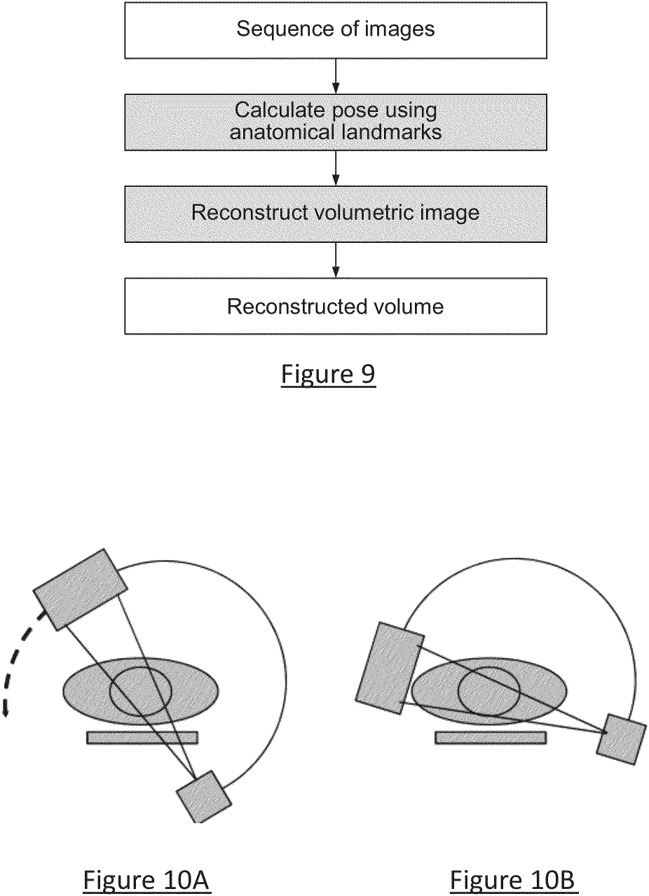

In some embodiments of the present invention, the subject (patient) anatomy can be used to extract a pose of every image using anatomical landmarks that are already part of the image. The non-limiting examples of such are ribs, lung diaphragm, trachea and others. This approach can be implemented by using 6-degree-of freedom pose estimation algorithms from 3D-2D correspondences. Such methods are also described in this patent disclose. See FIG. 9.

In some embodiments, the C-Arm movement continuity the missing frame poses can be extrapolated from the known frames. Alternatively, in such cases, a hybrid approach can be used through estimating a pose for a subset of frames through a pattern of radiopaque markers assuming that the pattern or its portion is visible for such computation.

In some embodiments, the present invention includes a pose estimation for every frame from the known trajectory movement of the imaging device assuming a trajectory of an X-ray imaging device is known or can be extrapolated and bounded. The non-limiting example of the FIG. 10A shows a pose of an X-ray imaging device mounted to a C-arm and covering a pattern of radiopaque markers. A subset of all frames having a pattern of radiopaque markers is used to estimate a 3D trajectory of the imaging device. This information is used to limit the pose estimation of pose of FIG. 10B to a specific 3D trajectory significantly limiting the solution search space.

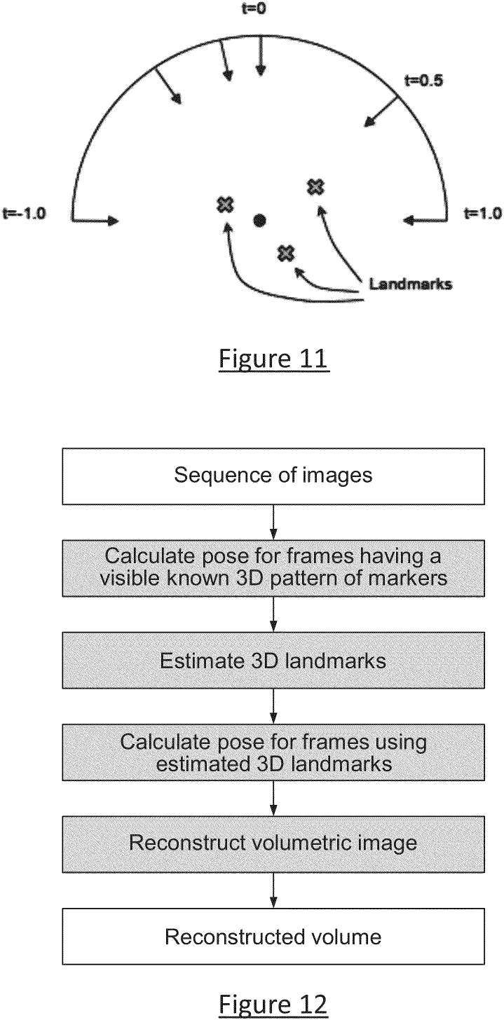

In some embodiments, after estimation of the 3D trajectory of a C-Arm movement, such movement can be represented by a small number of variables. In non-limiting example drawn by the Figure X1, the C-arm has such iso-center that 3D trajectory can be estimated using at least 2 known poses of a C-arm and it can be represented by a single parameter “t”. For this case, having at least one known and visible 3D landmark in the image is sufficient to estimate the parameter “t” in the trajectory corresponding to each pose of the C-Arm. See FIG. 11.

In some embodiments, in order to estimate the 3D position of landmarks at least two known poses of a C-arm are required using triangulation and assuming known intrinsic camera parameters. Additional poses can be used for more stable and robust landmarks position estimation.

In some embodiments, the method of performing tomographic volume reconstruction of the embodiment of the present invention, comprises:
Performing a rotation of an X-ray imaging device;
Estimating a trajectory of the X-ray imaging device movement using frames wherein estimated 3D landmarks are visible by solving a camera position and direction with 3D trajectory constraint and known 3D-2D correspond features;
Evaluation of the position on the trajectory of the frames where estimated 3D landmarks are invisible or partially visible through extrapolation algorithm based on an assumption of continuity of movement;
Estimating a pose of each frame by solving a camera position and direction with 3D trajectory constraint and known 3D-2D correspond features; and
Calculating volumetric reconstruction for the area of interest.

In some embodiments, the method of performing tomographic volume reconstruction of the present invention comprises:
Performing a rotation of an X-ray imaging device;
Estimating a trajectory of the X-ray imaging device using frames wherein pattern of radiopaque marker is visible and pose can be estimated;
Estimating a pose of frame where only estimated 3D landmarks are visible by solving a camera position and direction with 3D trajectory constraint and known 3D-2D correspond features; and
Calculating volumetric reconstruction for the area of interest.

In some embodiments, the present invention relates to a solution for the imaging device pose estimation problem without having any 2D-3D corresponding features (e.g. no prior CT image is required). Camera calibration process can be applied online or offline such as described by Furukawa, Y. and Ponce, J., “Accurate camera calibration from multi-view stereo and bundle adjustment,” International Journal of Computer Vision, 84(3), pp. 257-268 (2009) (which is incorporated herein by reference). Having a calibrated camera, structure from motion (SfM) technique can be applied to estimate the 3D structure of objects visible on multiple images. Such objects can be, but are not limited to, anatomical objects such as ribs, blood vessels, spine; instruments positioned inside a body such as endobronchial tools, wires, and sensors; or instruments positioned outside and proximate to a body, such as attached to the body; etc. In some embodiments, all cameras are solved together. Such structure from motion techniques are described in Torr, P.H. and Zisserman, A., “Feature based methods for structure and motion estimation. In International workshop on vision algorithms” (pp. 278-294) (September 1999), Springer, Berlin, Heidelberg (which is incorporated herein by reference).

In some embodiments, the present invention allows to overcome the limitation of using a known pattern of 3D radiopaque markers through the combination of target 3D pattern and the 3D landmarks that are being estimated dynamically from the time of the C-Arm rotation aimed to acquire imaging sequence for tomographic reconstruction or ever before such rotation. The non-limiting examples of such landmark are represented through objects either inside the patient’s body, such as markers on endobronchial tool, the tool tip, etc, or objects attached to the body exterior such as patches, wires etc.

In some embodiments, the said 3D landmarks are estimated using prior art tomography or stereo algorithms that utilize a visible and known set of radiopaque markers to estimate a pose per each image frame, as described in the FIG. 12.

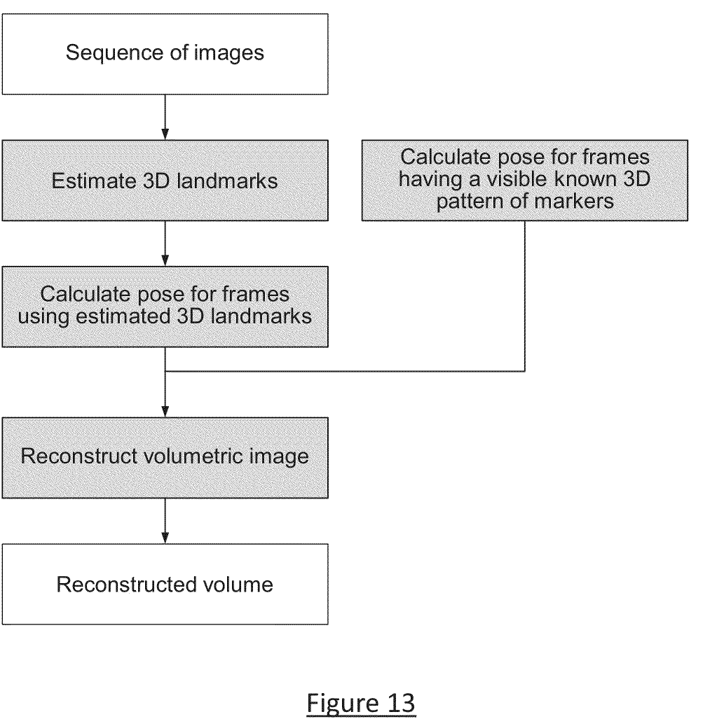

In some embodiments, alternatively, the said 3D landmarks are estimated using structure from motion (SfM) methods without relying on radiopaque markers in the frame as described in the FIG. 13. In the next step additional 3D landmarks are estimated. Poses for frames without known 3D pattern of markers are estimated with the help of estimated 3D landmarks. Finally, the volumetric reconstruction is computed using the sequence of all available images.

In some embodiments, the present invention is a method of reconstruction for three dimensional volume from a sequence of X-ray images comprising:
Estimating three dimensional landmarks from at least two frames with a known pose;
Using reconstructed landmarks to estimate a pose for other frames that do not have a pattern of radiopaque markers in the image frame; and
Calculating volumetric Reconstruction using all frames.

In some embodiments, present invention is an iterative reconstruction method that maximizes the output imaging quality by iteratively fine-tuning reconstruction algorithm input. As a non limiting example of image quality measurement might be measuring image sharpness. Because the sharpness is related to the contrast of an image and thus the contrast measure can be used as the sharpness or “auto-focus” function. The number of such measurements are defined in Groen, F., Young, I., and Ligthart, G., “A comparison of different focus functions for use in autofocus algorithms,” Cytometry 6, 81-91 (1985). As an example, the value ϕ(a) of the squared gradient focus measure for an image at area a is given by:

$$φ \left(\text{a}\right) = \sum \sum \sum \left(\text{f} \left(\text{x,y,z+1}\right) \text{-f} \left(\text{x,y,z}\right)\right) \text{\textasciicircum 2}$$

Since the area of interest should be roughly in the center of the reconstruction volume, it makes sense to limit the calculation to a small rectangular region in the center.

In some embodiments, this can be formulated as an optimization problem and solved using techniques like gradient descent.

Finetuning of poses happens by updating the function: pₙ+1=pₙ+α∇F(pₙ), where F denotes the reconstruction function given poses pₙ and then computing value of the sharpness function ϕ().

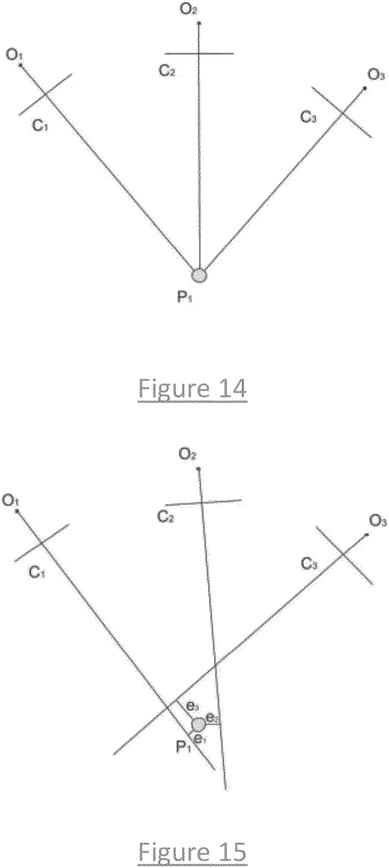

In some embodiments, the present invention is an iterative pose alignment method that improves the output imaging quality by iteratively fine-tuning camera poses to satisfy some geometric constraints. As a non-limiting example of such constraints can be a same feature point of an object visible in multiple frame and therefore have to be in the intersection of rays connecting that object and focal point of each camera (see FIG. 14).

Initially, most of the times this is not the case because of the inaccuracy in pose estimation and also due to displacement of the object (for instance, because of breathing). Correcting the poses of cameras to satisfy the rays intersection constraint will locally compensate for pose determination errors and movement of the imaged area of interest, resulting in better reconstruction image quality. Examples of such feature points could be the tip of the instrument inside the patient, or opaque markers on the instrument, etc.

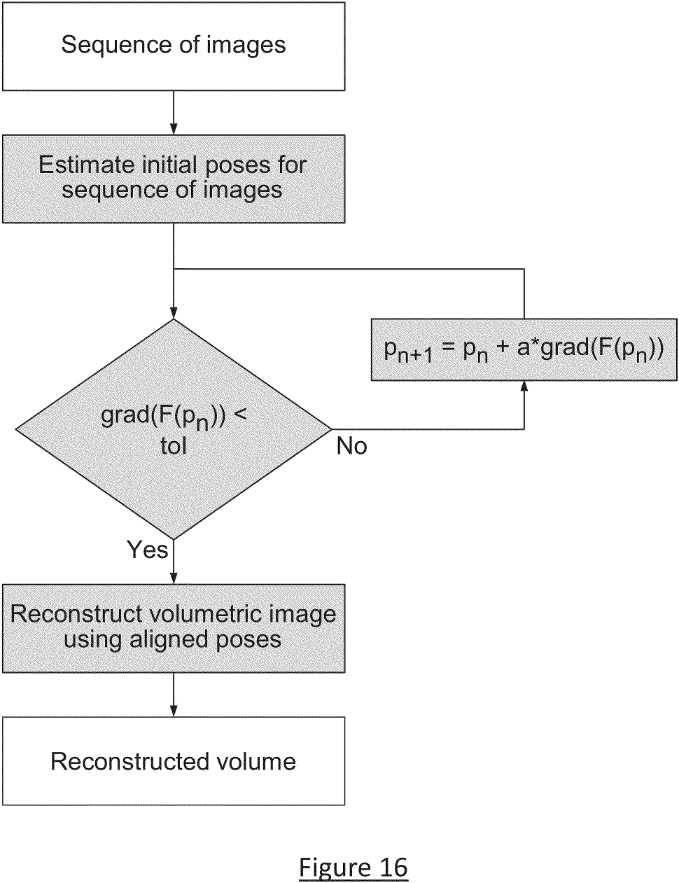

In some embodiments, this process can be formulated as an optimization problem and may be solved using methods such as gradient descent. See FIG. 16 for the method. The cost function can be defined as a sum of squared distances between object feature point and a closest point on each ray (see FIG. 15):

$$F \left(p\right) = {\sum }_{i} {e}_{i}^{2}$$

### Fluoroscopy Device Positioning Guidance

In some embodiments, each fluoroscope is calibrated before first usage. In some embodiments, a calibration process includes computing an actual fluoroscope rotation axis, registering preoperative and intraoperative imaging modalities, and displaying a target on an intraoperative image.

In some embodiments, before the C-arm rotation is started, the fluoroscope is positioned in a way that the target projected from preoperative image will remain in the center of the image during the entire C-Arm rotation.

In some embodiments, positioning the fluoroscope in such way that the target will be in the center of the fluoroscopic image is not, in and of itself, sufficient, as the fluoroscope height is critical, while the rotation center is not always in the middle of the image, causing undesired target shift outside the image area during the C-Arm rotation.

In some embodiments, as the target location is known relative to the reference system, an optimal 3D position of the C-Arm is calculated. In some embodiments, optimizing the 3D position of the C-Arm means minimizing the target’s maximal distance from the image center during the C-Arm rotation.

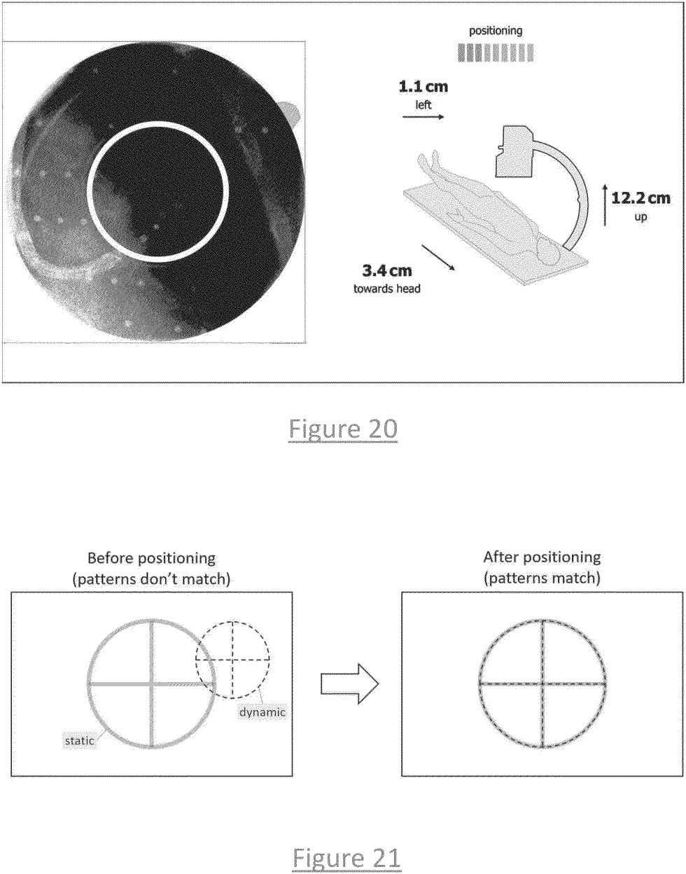

In some embodiments, to optimize the 3D position of the C-arm, a user first takes a single fluoroscopic snapshot. In some embodiments, based on calculations, the user is instructed to move the fluoroscope in 3 axes: up-down, left-right (relative to patient) and head-feet (relative to patient). In some embodiments, the instructions guide the fluoroscope towards its optimal location. In some embodiments, the user moves the fluoroscope according to the instructions and then takes another snapshot for getting new instructions, relative to the new location. FIG. 20 shows exemplary guidance that may be provided to the user in accordance with the above.

In some embodiments, for each snapshot, the location quality is computed by computing the percentage of the sweep (assuming +/- 30 degrees from AP) in which the lesion is entirely in the ROI, which is a small circle located in the image center.

In some embodiments, an alternative way to communicate the instructions is to display a static pattern and a similar dynamic pattern on an image, where the static pattern represents a desired location and the dynamic pattern represents a current target. In such embodiments, the user uses continuous fluoroscopic video and the dynamic pattern moves according to the fluoroscope movements. In some embodiments, the dynamic pattern moves in x and y axes according to fluoroscope’s movements in the left-right and head-feet axes, and the scale of the dynamic pattern changes according to fluoroscopy device movement in the vertical axis. In some embodiments, by aligning the dynamic and static patterns, the user properly positions the fluoroscopy device. FIG. 21 shows exemplary static and dynamic patterns as discussed above.

Example of Improved limited angle X-ray to CT reconstruction using unsupervised deep learning models.

There are different algorithms for 3D image reconstruction from 2D images, that receives as an input a set of 2D images of an object with a camera pose for every image and calculates a 3D reconstruction of the object. Those algorithms provides lower quality results when the 2D images are from limited angles (angle range of less than 180 degrees for X-rays) because of missing information. The proposed method results in considerable 3D image quality improvement in comparison to other methods that reconstruct the 3D image from limited angle 2D images.

In some embodiments, the present invention is an improved method that limited angle x-ray to CT reconstruction using unsupervised deep learning models, comprising:
Applying method of reconstruction low quality CT from x-ray images using existing method;
Applying image translation algorithm from domain A to domain C; and
Applying image translation algorithm from domain C to domain B.

For further discussion, the domains A, B and C will be used. Domain A, which is defined as “low quality tomographic reconstruction” domain, domain B, which is defined as a CT scan domain, domain C, which is defined as “simulated low quality tomographic reconstruction” generated from the pre-procedure CT data.

In some embodiments, section one can calculate pose for all the images, and then to reconstruct a low quality 3D reconstruction, for example, by the method “Using landmarks to estimate pose during tomography” described above, this method translates the 2D images to low quality CT image, inside domain A.

Continuing the last paragraph, the simulated low quality reconstruction, can be achieved by applying FP (forward projection) algorithm on a given CT, which calculates the intensity integrals along the selected CT axis and result in a simulated series of 2D X-ray images, the following step is applying method 1 from above, to reconstruct low quality 3D volume, for example by SIRT (Simultaneous Iterative Reconstruction Technique) algorithm, which iteratively reconstruct the volume, by starting with an initial guess of the result reconstruction, and iteratively applying FP and change the present reconstruction result by it’s FP difference from the 2D images (https://tomroelandts.com/articles/the-sirt-algorithm)

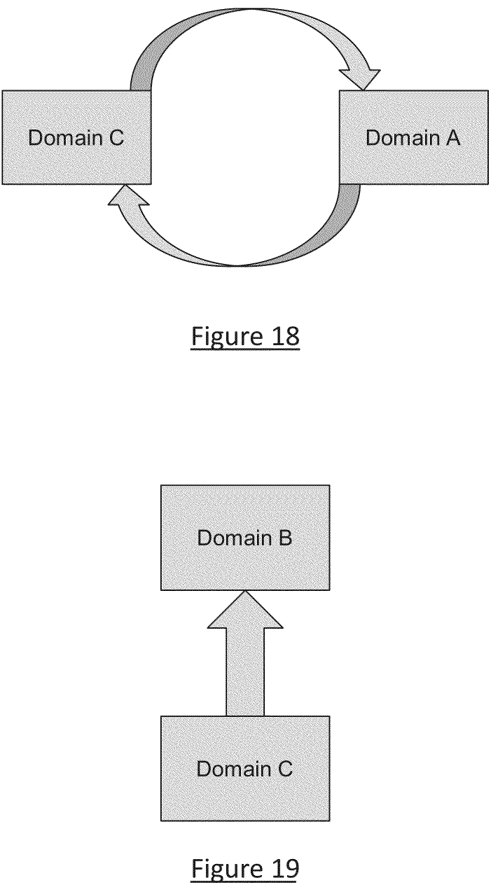

In some embodiments, the domain translation model that is used to translate a reconstruction from domain A to C, cannot be supervised (because the simulation is aligned to the CT, and there is not aligned CT for the 2D images). The simulated data can be produced by the method described above. It is possible to use Cycle-Consistent Adversarial Networks (Cycle Gan) to train the required model that translates reconstruction to his aligned simulation. The training of Cycle Gan, is done by combining adversarial loss, cycle loss, and identity loss (described at Jun-Yan Zhu, Taesung Park, Phillip Isola, and Alexei A Efros. 2017. Unpaired image-to-image translation using cycle-consistent adversarial networks. In Proceedings of the IEEE international conference on computer vision. 2223--2232.) which allows to train using unaligned images, as described in FIG. 18.

However, in some embodiments, the translation model from domain C to domain B can be supervised, because the creation of the simulation given a CT is aligned to the CT, by definition of the process. For example, i CNN-based neural network, with perceptual loss (as described in Justin Johnson, Alexandre Alahi, and Li Fei-Fei can be used. Perceptual losses for real-time style transfer and super-resolution. In ECCV, 2016) and L2 distance loss to train such model, as described in FIG. 19.

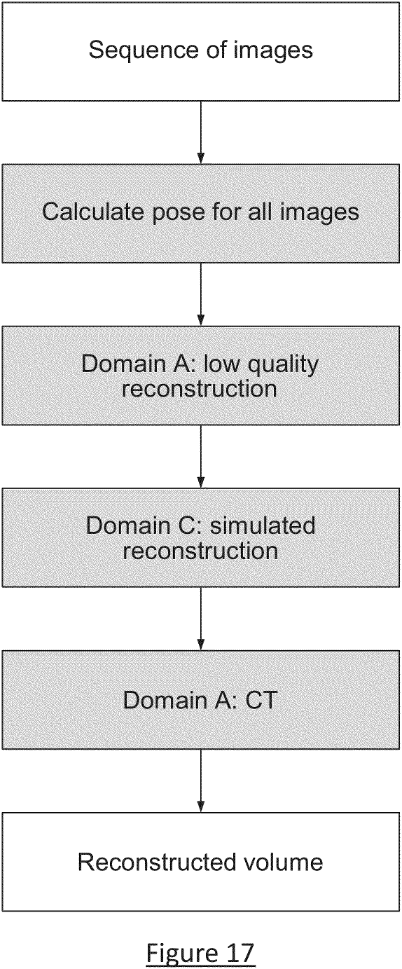

In some embodiments, the combination of all the methods described above appear in FIG. 17, which describes the process of starting with sequence of 2D images, and receives 3D image reconstruction

### EQUIVALENTS

The present invention provides among other things novel methods and compositions for treating mild to moderate acute pain and/or inflammation. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications, patents and sequence database entries mentioned herein are hereby incorporated by reference in their entireties as if each individual publication or patent was specifically and individually indicated to be incorporated by reference.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

What is claimed is:

1. A method, comprising:
receiving a sequence of medical images captured by a medical imaging device while the medical imaging device is rotated through a rotation, wherein the sequence of medical images show an area of interest that includes a plurality of landmarks;
determining a pose of each of a subset of the sequence of medical images in which the plurality of landmarks are visible;
estimating a trajectory of movement of the medical imaging device based on the determined poses of the subset of the sequence of medical images and a trajectory constraint of the imaging device;
determining a pose of at least one of the medical images in which the plurality of landmarks are at least partially not visible by extrapolating based on an assumption of continuity of movement of the medical imaging device; and
determining a volumetric reconstruction for the area of interest based at least on (a) at least some of the poses of the subset of the sequence of medical images in which the plurality of landmarks are visible and (b) at least one of the poses of the at least one of the medical images in which the plurality of landmarks are at least partially not visible.

2. The method of claim 1, wherein the poses of each of the subset of the sequence of medical images are determined based on 2D-3D correspondences between 3D positions of the plurality of landmarks and 2D positions of the plurality of landmarks as viewed in the subset of the sequence of medical images.

3. The method of claim 2, wherein the 3D positions of the plurality of landmarks are determined based on at least one preoperative image.

4. The method of claim 2, wherein the 3D positions of the plurality of landmarks are determined by application of a structure from motion technique.

5. A method, comprising:
receiving a plurality of medical images using an imaging device mounted to a C-arm while the medical imaging device is rotated through a motion of the C-arm having a constrained trajectory, wherein at least some of the plurality of medical images include an area of interest;
determining a pose of each of a subset of the plurality of medical images;
calculating locations of a plurality of 3D landmarks based on 2D locations of the 3D landmarks in the subset of the plurality of medical images and based on the determined poses of each of the subset of the plurality of medical images;
determining a pose of a further one of the plurality of medical images in which at least some of the 3D landmarks are visible by determining an imaging device position and an imaging device orientation based at least on a known 3D-2D correspondence of the 3D landmark; and
calculating a volumetric reconstruction of the area of interest based on at least the further one of the plurality of medical images and the pose of the further one of the plurality of medical images.

6. The method of claim 5, wherein the pose of each of the subset of the plurality of medical images is determined based at least on a pattern of radiopaque markers visible in the subset of the plurality of medical images.

7. The method of claim 6, wherein the pose is further determined based on the constrained traj ectory.

8. A method, comprising:
receiving a sequence of medical images captured by a medical imaging device while the medical imaging device is rotated through a rotation, wherein the sequence of medical images show an area of interest including a landmark having a 3D shape;
calculating a pose of each of at least some of the medical images based on at least 3D-2D correspondence of a 2D projection of the landmark in each of the at least some of the medical images; and
calculating a volumetric reconstruction of the area of interest based on at least the at least some of the medical images and the calculated poses of the at least some of the medical images.

9. The method of claim 8, wherein the landmark is an anatomical landmark.

10. The method of claim 9, wherein the 3D shape of the anatomical landmark is determined based at least on at least one preoperative image.

11. The method of claim 8, wherein the 3D shape of the landmark is determined based at least on applying a structure from motion technique to at least some of the sequence of medical images.

12. The method of claim 11, wherein the structure from motion technique is applied to all of the sequence of medical images.

13. The method of claim 8, wherein the pose is calculated for all of the sequence of medical images.

14. The method of claim 8, wherein the sequence of images does not show a plurality of radiopaque markers.

15. The method of claim 8, wherein the calculating a pose of each of the at least some of the medical images is further based on a known trajectory of the rotation.

16. The method of claim 8, wherein the 3D shape of the landmark is determined based on at least one preoperative image and further based on applying a structure from motion technique to at least some of the sequence of medical images.

17. The method of claim 8, wherein the landmark is an instrument positioned within a body of a patient at the area of interest.

18. The method of claim 8, wherein the landmark is an object positioned proximate to a body of a patient and outside the body of the patient.

19. The method of claim 18, wherein the object is fixed to the body of the patient.
